(19) United States
(12) Patent Application Publication
(10) Pub. No.: US 2008/0146874 A1
(43) Pub. Date: Jun. 19, 2008

(54) Guiding stylet apparatus

(76) Inventor: Chieh-Hsiao Chen, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/790,103

(22) Filed: Apr. 24, 2007

(30) Foreign Application Priority Data
Dec. 13, 2006 (TW) 95146758

(51) Int. Cl. A61B 1/04

(52) U.S. Cl. 600/109; 600/104; 600/108

(57) ABSTRACT

A guiding stylet apparatus is disclosed. The guiding stylet apparatus comprises a guiding device, a holding portion, an image capture unit, a light source, and a display. The guiding device is inserted into a catheter to guide the catheter to a predetermined position. The image capture unit is used to capture an internal image. The light source provides sufficient light, so as to allow the image capture unit to capture the internal image. The display is electrically connected to the image capture unit to display the internal image.

### RELATED APPLICATIONS

This application claims priority to Taiwan Application Serial Number 95146758, filed Dec. 13, 2006, which is herein incorporated by reference.

### FIELD OF THE INVENTION

This invention relates to a guiding stylet apparatus, and more particularly, to an apparatus that guides a catheter when the catheter is implanted.

### BACKGROUND OF THE INVENTION

In modern medical treatment, occasionally, it is necessary to create a passageway from the inside of the human body to the outside to perform specific medical treatments, such as implantation of continuous ambulatory peritoneal dialysis (CAPD) catheter to implant a continuous ambulatory peritoneal dialysis catheter into the body of a patient, so as to perform continuous ambulatory peritoneal dialysis (CAPD); or oral endotracheal tube insertion to insert an endotracheal tube through the mouth thereby keeping the airway of the patient unobstructed and further providing oxygen. Taking the implantation of CAPD catheter for example, a CAPD catheter is implanted by surgery. First, the abdomen of the patient is cut open to open an entrance. Then the CAPD catheter is implanted into the abdomen cavity of the patient from the entrance with a stylet to guide the catheter, wherein the opening of the CAPD catheter must be located at the bottom of the abdomen cavity to perform CAPD. However, the guiding of the catheter sometimes only depends on the doctor's experience and judgments. Therefore, the implanting position of the catheter may be improper and lead to a failure of the implantation of the catheter. Sometimes, the adhesion in the abdomen cavity of the patient may fail the implantation of the catheter, but the doctor cannot immediately monitor the status of the implantation in surgery. Sometimes, the implantation of the catheter is performed with an endoscope, but at least two or three wounds for trocars have to be created on the abdomen of the patient to insert the endoscope or other operation implements. Therefore, the wounds made by the surgeon increase, and thus affect the post-operation healing, increase rate of complication, and raise the risk of anesthesia.

### SUMMARY OF THE INVENTION

Therefore, an aspect of the present invention is to provide a guiding stylet apparatus to monitor the position of a catheter inside the human body immediately, thereby preventing the catheter from being implanted in the incorrect position.

Another aspect of the present invention is to provide a guiding stylet apparatus to monitor the position of a catheter inside the human body immediately, thereby preventing the need for additional monitoring apparatus to be inserted in the human body.

According to an embodiment of the present invention, the guiding stylet apparatus comprises a guiding device, a holding portion, an image capture unit, a light source, and a display. The guiding device is inserted into a catheter, wherein the guiding device has a curved stick-type structure to guide the catheter to a predetermined position. The holding portion is disposed at one end of the guiding device for holding. The image capture unit is disposed at another end of the guiding device to capture an internal image. The light source is disposed on the guiding device to provide sufficient light, so as to allow the image capture unit to capture the internal image. The display is electrically connected to the image capture unit to display the internal image.

According to another embodiment of the present invention, the curved stick-type structure of the guiding device is adjustable, and the display is disposed on the holding portion to display to display the internal image.

Therefore, with the application of the guiding stylet apparatus disclosed in the embodiments of the present invention, the catheter can be guided to the proper implanting position. In the implanting process, doctors can monitor the position of the catheter inside the human body immediately and make necessary positional adjustments when guiding catheter during the implantation process, and thereby preventing the catheter from being implanted at the incorrect position. The guiding stylet apparatus of the present invention can monitor the position of the catheter inside the human body without inserting other monitoring apparatus, so as to reduce unnecessary wounds and discomfort made by surgery.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a schematic front view showing a guiding stylet apparatus according to a first embodiment of the present invention;

FIG. 2 is a schematic front view showing a guiding stylet apparatus inserted in a catheter according to a first embodiment of the present invention;

FIG. 3 is a cross-sectional view showing a guiding stylet apparatus in guiding according to a second embodiment of the present invention;

FIG. 4 is a schematic front view showing a guiding stylet apparatus according to a second embodiment of the present invention;

FIG. 5 is a schematic front view showing a guiding stylet apparatus according to a third embodiment of the present invention;

FIG. 6 is a schematic front view showing a guiding stylet apparatus according to a fourth embodiment of the present invention;

### DETAILED DESCRIPTION OF THE PREFERRED EMBODIMENT

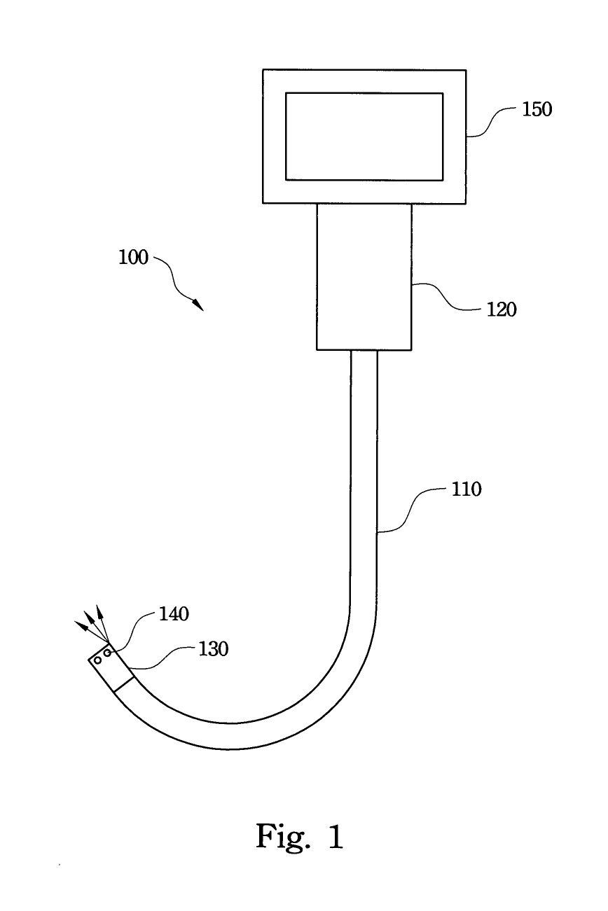

In order to make the illustration of the present invention more explicit and complete, the following description is stated with reference to FIG. 1 through FIG. 6.

The guiding stylet apparatus 100 of the present invention is used to guide a catheter 200 in the human body and monitor the position of the catheter immediately when a catheter is being implanted, thereby implanting the catheter 200 into a proper position.

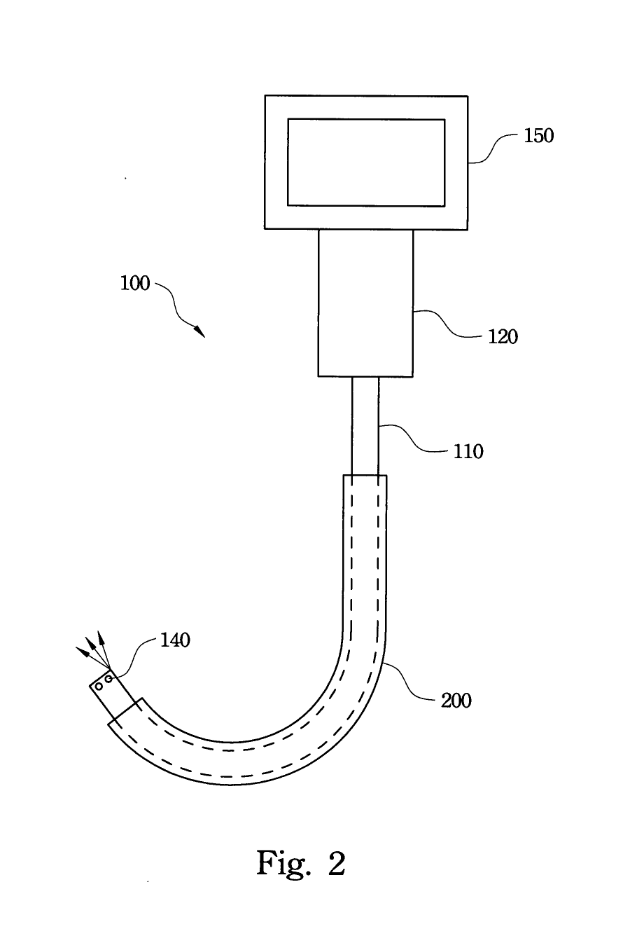

Refer to FIG. 1 and FIG. 2. FIG. 1 is a schematic front view showing a guiding stylet apparatus according to a first embodiment of the present invention, and FIG. 2 is a schematic front view showing a guiding stylet apparatus inserted in a catheter according to a first embodiment of the present invention. The guiding stylet apparatus 100 of the first embodiment comprises a guiding device 110, a holding portion 120, an image capture unit 130, a light source 140 and a display 150. The guiding device 110 is inserted into a catheter 200 to guide the catheter 200 to a predetermined position. The catheter 200 is such as a continuous ambulatory peritoneal dialysis (CAPD) catheter or an endotracheal tube. The holding portion 120 is used for holding, so that doctor can hold the holding portion 200 to adjust the position of the catheter 200. The image capture unit 130 captures an internal image of the human body so that doctor can watch the position of the catheter 200 inside the body. The light source 140 provides sufficient light, so as to allow the image capture unit 130 to capture the internal image clearly. The display 150 displays the internal image captured inside the human body, so that the doctor can monitor the position of the catheter 200 inside the human body immediately from the display 150.

Refer to FIG. 1 and FIG. 2 again. The guiding device 110 of the first embodiment has a curved stick-type structure that can be inserted into the catheter 200. The curved stick-type structure corresponds to the human body structure to guide the catheter 200 to the predetermined position inside the human body, such as using the curved stick-type structure of the guiding device 110 to guide the catheter 200 implant to the bottom of the abdominal cavity of the human body to perform continuous ambulatory peritoneal dialysis (CAPD). The guiding device 110 is a tube-like structure to provide the electrical disposing space and the light passageway, and further the guiding device 110 can have a hollow and multi-channel structure for multiple purposes, such as implement (like laser knife) inserting, or a passageway to provide gas (like carbon dioxide) into the human body. The holding portion 120 is disposed at one end of the guiding device 110 for holding, whereby the doctor can hold the holding portion 120 to adjust the position of the guiding stylet apparatus 100, so as to guide the catheter 200 to the predetermined position. The image capture unit 130 may be a charge-coupled device (CCD) disposed at another end of the guiding device 110 to capture the internal image of the human body and transmit the internal image to the display 150, whereby the doctor can monitor the position of the catheter 200 immediately from the display 150 and hold the holding portion 120 to guide the catheter 200. The light source 140 may be a light emitting diode (LED) to provide sufficient light, so as to allow the image capture unit 130 to capture the internal image. The light source 140 is disposed at another end of the guiding device 110 and close to the image capture unit 130 to light the inside of the human body directly.

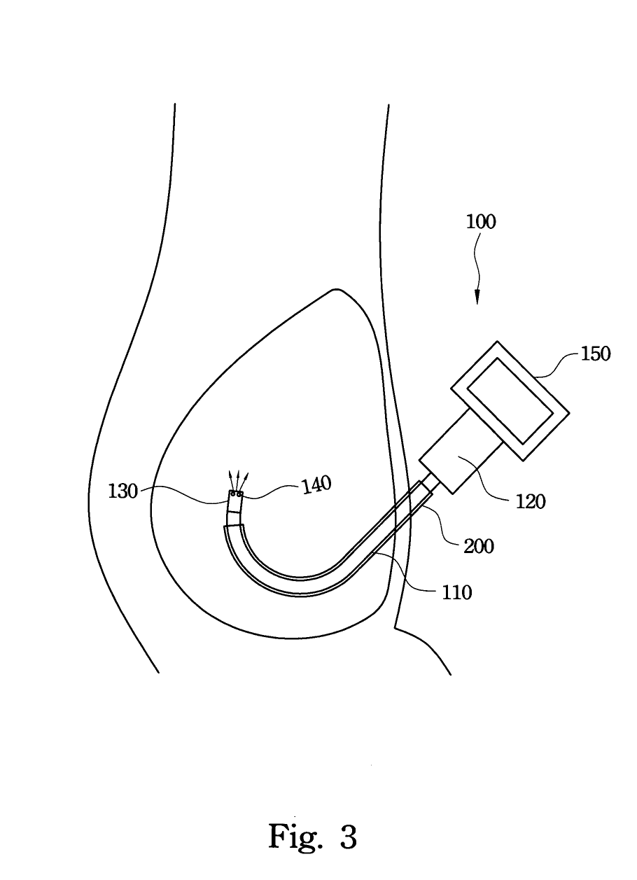

Refer to FIG. 3. FIG. 3 is a cross-sectional view showing a guiding stylet apparatus guiding the catheter in accordance with the second embodiment of the present invention. The display 150 may be a liquid crystal display (LCD) electrically connected to the image capture unit 130 to display the internal image. In the first embodiment of the present invention the display 150 is a small-scale display disposed on the holding portion 120 and positioned to make watching the screen convenient for the doctor. Therefore, when implanting the catheter 200, the doctor can easily monitor the position of the catheter 200 immediately and adjust the implanting position of the catheter 200 inside the human body.

Refer to FIG. 3 again. When using the guiding stylet apparatus 100 of the first embodiment to guide the catheter 200, at first, the guiding device 110 can be inserted into the catheter 200. Next, the doctor can hold the holding portion 120 to insert the guiding stylet apparatus 100 into the human body and use the curved stick-type structure of the guiding device 110 to guide the implanting position of the catheter 200, wherein the entrance that allows the guiding stylet apparatus 100 to be inserted into the human body is created by surgery (such as the implantation of CAPD catheter), and can also be a inherent opening of the human body (such as the mouth or the nostril). Next, the doctor can watch the internal image captured by the image capture unit 130 from the display 150 to monitor the position of the catheter 200 immediately and hold the holding portion 120 to adjust the guiding of the catheter 200 to position the catheter 200 at the predetermined position. Then, after positioning the catheter 200 at the predetermined position, the guiding stylet apparatus 100 is withdrawn from the human body, thereby implanting the catheter 200 at the predetermined position inside the human body, wherein one end of the catheter 200 is located inside the human body, and another end of the catheter 200 is located outside the human body after implanting the catheter 200. Hence, the catheter 200 implanted into the human body can be a passageway from inside the human body to the outside to enable specific medical treatments, such as CAPD or providing oxygen to be performed. Therefore, the guiding stylet apparatus 100 of this embodiment monitors the position inside the human body immediately and guiding the catheter 200 to the predetermined position at the same time without inserting other monitor apparatus, thereby avoiding the discomfort made by surgery and unnecessary wounds to insert other monitoring apparatus. Besides, the guiding stylet apparatus 100 can straight be used for being an endoscope to watch the internal status of the human body without inserting into the catheter 200.

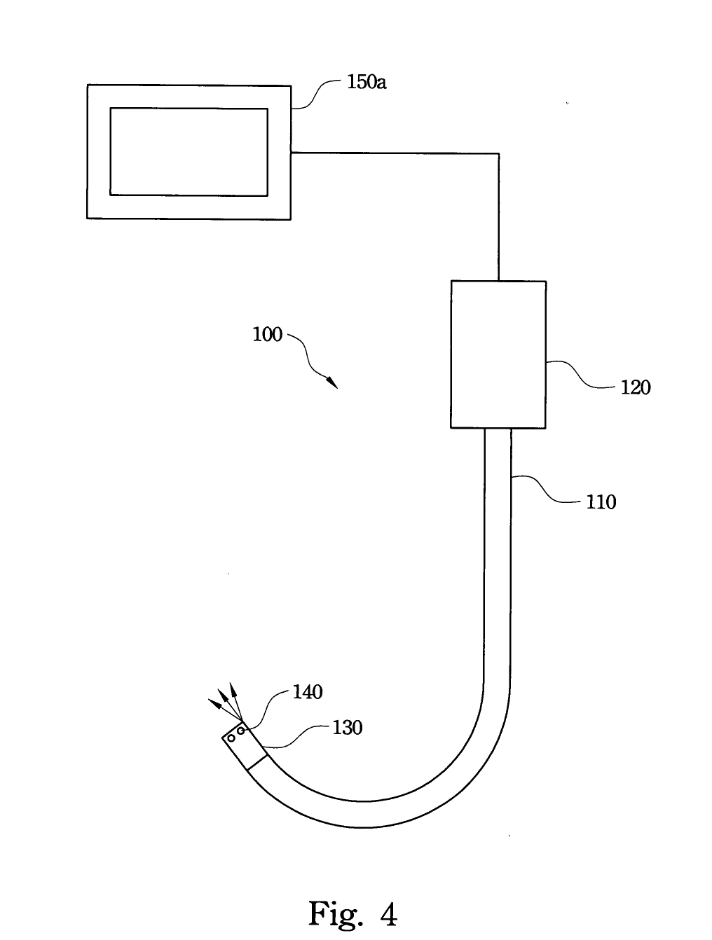

Refer to FIG. 4. FIG. 4 is a schematic front view showing a guiding stylet apparatus according to a second embodiment of the present invention. Same reference numerals shown in FIG. 1 are used in the second embodiment of the present invention. The construction of the guiding stylet apparatus structure shown in the second embodiment is similar to that in the first embodiment with respect to configuration and function, and thus is not stated in detail herein.

Refer again to FIG. 4. In comparison with the first embodiment, the display 150a of the guiding stylet apparatus 100 of the second embodiment is not disposed on the holding portion 120, but separately disposed at a position convenient to watch and electrically connected to the image capture unit 130. Therefore, the guiding stylet apparatus 100 of the second embodiment is easier to hold, thereby making it easier to guide the catheter 200.

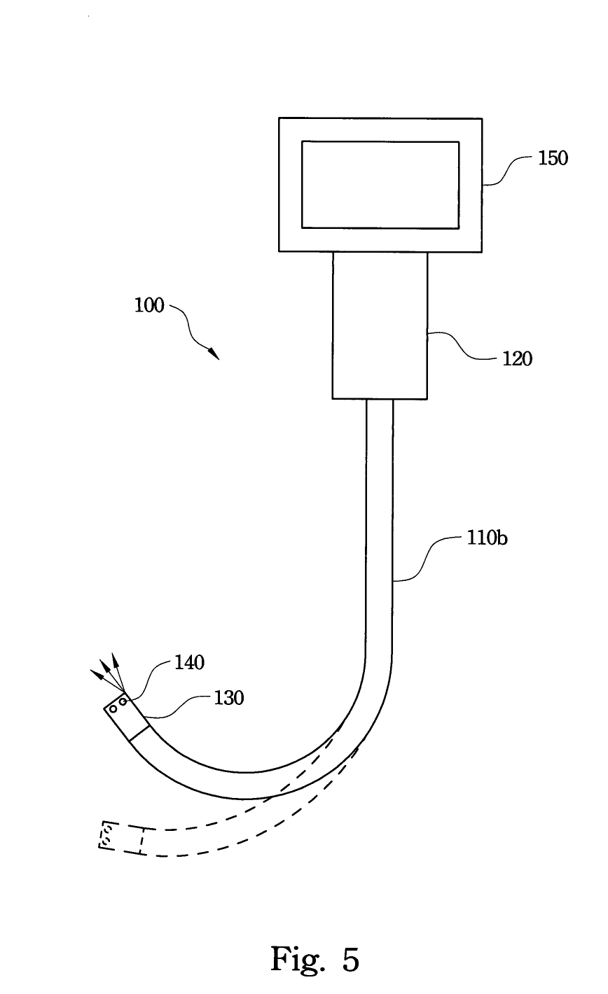

Refer to FIG. 5. FIG. 5 is a schematic front view showing a guiding stylet apparatus according to a third embodiment of the present invention. Same reference numerals shown in FIG. 1 are used in the third embodiment of the present invention. The construction of the guiding stylet apparatus structure shown in the third embodiment is similar to that in the first embodiment with respect to configuration and function, and thus is not stated in detail herein.

Refer again to FIG. 5. In comparison with the first embodiment, the guiding device 110b of the guiding stylet apparatus 100 of the fourth embodiment has a specific flexibility to bend, thereby adjusting the curved stick-type structure of the guiding device 110b. Therefore, the curved stick-type structure of the guiding device 110b can be adjusted corresponding to the real using situation, thereby increasing the using margin of the guiding stylet apparatus 100 of the third embodiment.

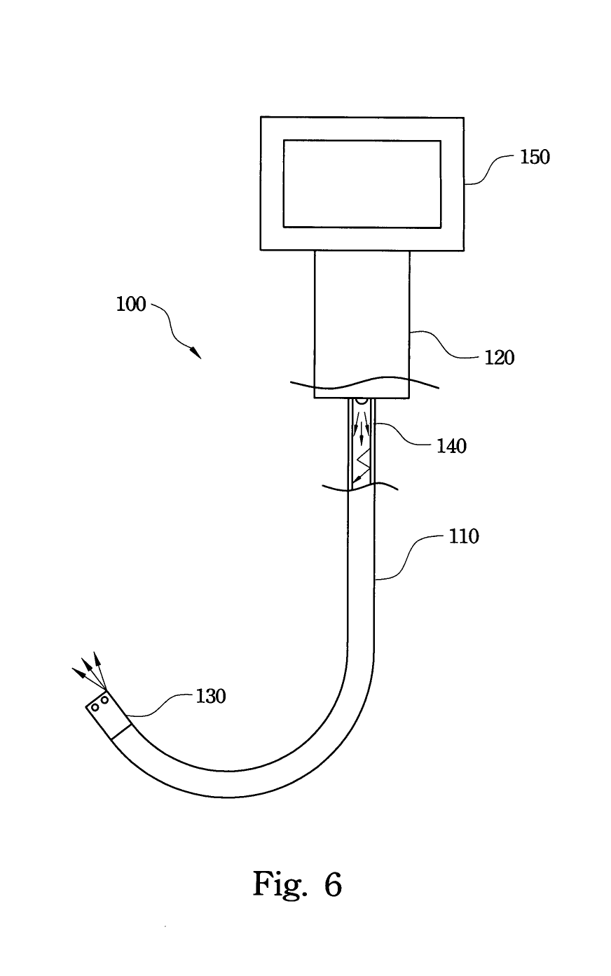

Refer to FIG. 6. FIG. 6 is a schematic front view showing a guiding stylet apparatus according to a fourth embodiment of the present invention. Same reference numerals shown in FIG. 1 are used in the fourth embodiment of the present invention. The construction of the guiding stylet apparatus structure shown in the fourth embodiment is similar to that in the first embodiment with respect to configuration and function, and thus is not stated in detail herein.

Refer again to FIG. 6. In comparison with the first embodiment, the light source 140c of the guiding stylet apparatus 100 of the fourth embodiment is disposed at one end of the guiding device 110 and closed to the holding portion 120, and an optical fiber module 111 is disposed in the tube-like structure of the guiding device 110. Therefore, the light emitted from the light source 140c can be transmitted into the human body through the optical fiber module 111 for lighting.

Therefore, the guiding stylet apparatus shown in the respective embodiments of the present invention monitors the position of the catheter inside the human body immediately and guides the catheter, thereby preventing the catheter from being implanted in the wrong position and enhancing the medical quality. Further, the guiding stylet apparatus of the present invention monitors the position inside the human body without inserting other monitor apparatus, so as to reduce unnecessary wounds and discomfort made by surgery.

As is understood by a person skilled in the art, the foregoing embodiments of the present invention are strengths of the present invention rather than limiting of the present invention. It is intended to cover various modifications and similar arrangements included within the spirit and scope of the appended claims, the scope of which should be accorded the broadest interpretation so as to encompass all such modifications and similar structure.

What is claimed is:

1. A guiding stylet apparatus, comprising:
a guiding device inserted into a catheter, wherein the guiding device has a curved stick-type structure to guide the catheter to a predetermined position;
a holding portion disposed at one end of the guiding device for holding;
an image capture unit disposed at another end of the guiding device for capturing an internal image;
a light source disposed on the guiding device to provide sufficient light, so as to allow the image capture unit to capture the internal image; and
a display electrically connected to the image capture unit to display the internal image.

2. The guiding stylet apparatus as claimed in claim 1, wherein the curved stick-type structure of the guiding device is adjustable.

3. The guiding stylet apparatus as claimed in claim 1, wherein the image capture unit is a charge-coupled device (CCD).

4. The guiding stylet apparatus as claimed in claim 1, wherein the display is a liquid crystal display (LCD).

5. The guiding stylet apparatus as claimed in claim 1, wherein the catheter is a continuous ambulatory peritoneal dialysis (CAPD) catheter.

6. The guiding stylet apparatus as claimed in claim 1, wherein the catheter is an oral endotracheal tube.

7. The guiding stylet apparatus as claimed in claim 1, wherein the light source is a light emitting diode (LED).

8. The guiding stylet apparatus as claimed in claim 1, wherein the light source is disposed at the end of the guiding device.

9. The guiding stylet apparatus as claimed in claim 1, wherein the light source is disposed at the other end of the guiding device.

10. The guiding stylet as claimed in claim 1, wherein the guiding device further has a tube-like structure, and a fiber module is disposed in the tube-like structure of the guiding device.

11. The guiding stylet apparatus as claimed in claim 1, wherein the guiding device further has a hollow multi-channel structure to dispose at least one implement.

12. The guiding stylet apparatus as claimed in claim 11, wherein the implement is a laser knife.

13. The guiding stylet apparatus as claimed in claim 1, wherein the display is disposed on the holding portion.

14. The guiding stylet apparatus as claimed in claim 1, wherein the guiding stylet apparatus is used for being an endoscopy.

15. A guiding stylet apparatus, comprising:
a guiding device inserted into a catheter, wherein the guiding device has a curved stick-type structure to guide the catheter to a predetermined position, and the curved stick-type structure of the guiding device is allowed to be adjusted;
a holding portion disposed at one end of the guiding device for holding;
an image capture unit disposed at another end of the guiding device for capturing an internal image;
a light source disposed on the guiding device to provide sufficient light, so as to allow the image capture unit to capture the internal image; and
a display disposed on the holding portion and electrically connected to the image capture unit to display the internal image.
